(19) United States
(12) Patent Application Publication
(10) Pub. No.: US 2002/0019036 A1
(43) Pub. Date: Feb. 14, 2002

(54) Von willebrand factor derivatives and methods of isolating proteins that bind to von willebrand factor

(76) Inventors: Hans-Peter Schwarz, Vienna (AT); Peter Turecek, Klosterneuburg (AT); Johann Eibl, Vienna (AT)

Correspondence Address: HELLER EHRMAN WHITE & MCAULIFFE LLP, 1666 K STREET,NW, SUITE 300, WASHINGTON, DC, 20006, US

(21) Appl. No.: 09/967,937

(22) Filed: Oct. 2, 2001

Related U.S. Application Data
(62) Division of application No. 09/319,116, filed on Jun. 2, 1999.
Application No. 09/319,116 is a 371 of international application No. PCT/AT97/00253, filed on Nov. 19, 1997.

(30) Foreign Application Priority Data
Dec. 13, 1996 (AT) A 2178/96

(51) Int. Cl. C12N 11/16; C07K 14/755

(52) U.S. Cl. 435/174; 530/383

(57) ABSTRACT

There is disclosed a vWF derivative comprised of vWF, immobilized on a carrier, which is characterized in that the vWF is r-vWF, as well as a method of isolating proteins which bind to vWF, by using this vWF derivative.

The invention relates to a von Willebrand factor derivative as well as to a method of isolating proteins binding to vWF.

von Willebrand factor (vWF) is a glycoprotein circulating in plasma in a series of multimers ranging in size from approximately 500 to 20 000 kilodaltons. The multimeric forms of vWF are assembled of 250 kD polypeptide sub-units bound to each other by disulfide links. vWF is involved in the binding of blood platelets to the subendothelium of a damaged vessel wall, wherein only the larges multimeres also exhibit a hemostatic activity. It is assumed that the endothelium cells secrete large polymeric forms of vWF and that those forms of vWF which have a lower molecular weight (low molecular weight vWF, LMW) have formed by proteolytic cleavages.

In the vascular endothelium cells which constitute the main source of this plasma protein, vWF is formed by constitutive or stimulated liberation, yet in a smaller portion it is also synthesized by the megakaryocytes. The biosynthesis of vWF is very complex and thus results in a plurality of the most varying vWF molecules of the most different structures, tasks and properties.

As a consequence, vWF can bind to various receptors in different tissues, its binding to proteins, such as glycoprotein Ib, the glycoprotein complex II/IIIa, factor VIII:C, to thrombocytes, to the subendothelium being among the most important physiological activities of vWF.

When vWF is bound to blood coagulation factor VIII, the factor VIII complex or factor VIII:C/vWF-complex is formed which contains factor VIII:C as stabilized protein. vWF deficiency of necessity will also lead to a reduction of the factor VIII:C concentration in blood, since the stabilizing effect of vWF is missing.

It is known to purify recombinant factor VIII by means of a column containing plasmatic von Willebrand factor coupled to Sepharose® (vWF-Sepharose) (Wood et al., Nature 312 (1984), 330-337). It has, however, been shown that the avidity of an immobilized plasmatic vWF is low, and thus, e.g., the recovery of factor VIII from a solution containing factor VIII/vWF-complex by means of the vWF-Sepharose described is not feasible in note-worthy yields.

Therebeyond, besides vWF, this vWF-Sepharose also contained factor VIII because of the plasmatic starting material which contains the factor VIII:C/vWF-complex. In the recovery of pharmaceutical preparations, however, the contamination with plasmatic factor VIII:C would constitute a major problem.

Binding studies examining the inhibition of the binding of factor VIII to plasmatic vWF immobilized in the wells of microtiter plates, by means of recombinant vWF, furthermore have shown that with recombinant vWF in solution an even poorer avidity regarding its binding partner factor VIII:C could be attained (Leyte et al., Biochem. J., 274 (1991), 257-261). For this reason, this material is not suitable for a preparative method of producing factor VIII.

Thus, it is the object of the present invention to provide a chromatographic material by which proteins binding to vWF can be isolated even if vWF is present in the solution from which the proteins are to be isolated. In other words, the object of the present invention is to provide a material having an avidity which is higher than that of vWF when bound to its binding partners, in particular higher than that of the vWF in the factor VIII/vWF-complex.

According to the invention, this object is achieved by a vWF derivative comprised of vWF, immobilized on a particulate carrier or a carrier gel (carrier), in particular on a chromatographic material which is characterized in that the vWF is a recombinant vWF (r-vWF). Preferably, the vWF derivative is free from blood coagulation factor VIII, and because of the avoidance of anti-vWF-antibodies, free from xenogenic material, such as antibodies.

Surprisingly, it has been shown that—in contrast to plasmatic vWF—r-vWF has a surprisingly high avidity relative to vWF-binding proteins, although it has been bound to a chromatographic material.

Moreover, in contrast to plasmatic vWF, r-vWF is free from blood coagulation factor VIII, and in particular free from plasmatic proteins which would be troublesome in an isolation procedure for vWF-binding proteins.

In a particular embodiment of the vWF derivative according to the invention, the r-vWF is fixed to the chromatographic material by chemical binding. In this manner it is ensured that the high avidity of the vWF derivative of the invention is maintained with high stability, which enormously increases the applicability of the material of the invention. By the chemical fixation, stabilization of the chromatographic material has been attained, while, surprisingly, the native structure of vWF has not been negatively affected. Simultaneously, the use of corresponding antibodies which confer a binding of the vWF to a solid phase can be omitted. A binding via antibodies would have the disadvantage of instability and an increased “leakage”, i.e. dissolving out of the immobilized vWF simultaneously with the recovery of the binding partners for vWF.

The avidity of the vWF derivative can be further increased by a careful selection of the r-vWF fraction. Surprisingly, it has been found that particularly from an r-vWF fraction of low primary hemostatic activity, in particular from a low-molecular r-vWF fraction having a molecular weight of less than approximately 1.5 million Da, preferably less than 1 million Da, a material of high avidity for factor VIII can be prepared.

The availability of recombinant von Willebrand factor in high purity and unlimited quantity allows for a wide application of the inventive vWF derivative as a ligand in affinity chromatography or related affinity methods, in which a molecule to be bound is bound by a specific interaction with the ligand.

r-vWF is preferably expressed in mammalian cells, e.g. CHO cells. Such a vWF has been described in Patent Application WO 96/10584. When purifying r-vWF, e.g. via heparin affinity chromatography, fractions are incurred which contain molecules having a molecular weight which is relatively low for vWF (up to approximately 1 million Dalton). Although these fractions as such-have a relatively low primary hemostatic activity, they are, however, still capable to bind binding proteins which enter into specific interactions with vWF, e.g. factor VIII. Since these secondary fractions of the r-vWF production are not used for therapeutically applied vWF concentrates comprising a large portion of high multimers, these constitute a secondary product which, according to the invention, can be used for immobilization and subsequent use of the immobilisate for preparing an affinity matrix.

As the chromatographic material, according to the invention, preferably a gel is used which has good affinity chromatographic properties, i.e. a slight back pressure corresponding to high flow rates, a high binding capacity for the ligand to be immobilized, a low leakage behavior and the possibility for disinfection, e.g. by means of a soda lye.

Coupling of the r-vWF to the solid matrix is carried out such that the binding properties for the proteins to be bound in the affinity chromatography do not get lost. For this purpose, surprisingly, standard immobilization techniques can be used, such as, e.g., described in Woodward “Immobilized Cells and Enzymes”, IRL. Press, Oxford, Wash. (1985), pp.3-17. Furthermore, such a chromatographic gel has good reutilization properties and stability so that also in case of repeated use, the binding capacity for the molecules to be isolated remains constant.

A preferred vWF derivative thus comprises an organic polymer, in particular an organic polymer based on carbohydrates, as the chromatographic material. Suitable gel matrices or particulate carriers, respectively, are, e.g., Sephacels®, Sephadexes®, Sepharoses®, fast flow media, high performance media, Sepharose Big Beads® (all from Pharmacia), Tris-Acryl®-, Spherodex®- or Hyper-D®-media (all from Sepracor), Macropep® gels (BioRad), yet also synthetic polymers, such as Toyopearl® gels (Tosohaas) and Fractogels® (Merck).

To avoid contaminations, in particular with human pathogenic viruses, the vWF derivative of the invention preferably is subjected to a virus inactivation treatment so that it can be ensured that the chromatographic material does not constitute a virus contamination factor when proteins which bind to vWF are isolated. The treatment for the inactivation of viruses preferably is carried out prior to derivatisation, by a chemical and/or physical method, e.g. by a treatment with tensides, polyethylene glycols, chaotropic substances, by a heat treatment or a radiation treatment. Likewise, however, also the finished derivative, preferably in lyophilized form, may be subjected to a treatment, such as the heat treatment or radiation treatment.

In a preferred embodiment, the vWF derivative of the invention is provided in storage-stable form, in particular as a lyophilisate, whereby their trading, sale and storage are considerably facilitated.

According to a further aspect, the present invention relates to a device comprising a container and a vWF derivative according to the invention contained therein, the container comprising an inlet aperture and an outlet aperture which are designed to be suitable for passing liquids therethrough. Practically, the device according to the invention is designed as a column, in particular an affinity column or a chromatographic column, which, if desired, may be provided as a ready-to-use product in storage-stable form, so that merely swelling of the material must be carried out by the respective user prior to protein isolation.

The present invention also relates to a method of isolating proteins which bind to vWF and which is characterized by the following steps:

providing a fraction containing proteins which bind to vWF,

contacting the fraction with a vWF derivative of the invention, wherein the proteins become bound to the vWF derivative,

separating non-bound components, and

eluting the proteins from the vWF derivative.

This method may be carried out batch-wise, or it may be carried out as a column-chromatographic method.

As proteins which may be isolated with the method of the invention, primarily the physiological binding proteins of vWF are under consideration, i.e. glycoprotein Ib, the glycoprotein IIb/IIIa-complex, collagen, and in particular factor VIII, yet, of course, also recombinant derivatives and analogues of these proteins, vWF antigens, vWF antibodies, vWF multimerases or vWF depolymerases, respectively, and even enzymes which recognize vWF as substrate, and other natural or synthetic peptides and proteins which have an affinity to vWF. Besides, vWF-binding saccharides, such as, e.g., heparin, can be isolated by this method.

Due to the high avidity of the vWF derivative according to the invention it is possible to specifically bind the proteins to be isolated to the inventive chromatographic material and to recover them in yields of more than 60%, preferably more than 80%, most preferred nearly quantitatively, and to elute them from the vWF derivative inpurified form, thus enabling the preparation of concentrates of the isolated proteins by simple elution without a subsequent concentration step.

The method according to the invention is particularly suitable for recovering biologically active proteins having factor VIII activity, in particular of plasmatic or recombinant factor VIII and the mutants or analogues thereof, respectively. It may be isolated with the method of the invention even if a starting solution comprising factor VIII/vWF-complex is used.

Elution of the proteins, in particular in the recovery of factor VIII proteins, preferably is carried out with a calcium-ion-containing buffer.

In doing so, what is particularly important is the isolation of factor VIII from factor VIII-containing plasma fractions or from cell culture supernatants of cells which express factor VIII. When isolating factor VIII from plasma fractions, care must be taken that vWF functions as the carrier protein of factor VIII, i.e. that factor VIII occurs bound to vWF. The separation of factor VIII and vWF otherwise can be effected only with complex methods, e.g. by binding factor VIII to immobilized mono- or polyclonal antibodies directed against factor VIII, to which the factor VIII/vWF-complex binds, whereupon vWF subsequently is purposefully eluted without disturbing the bond of the antibody with factor VIII. Thereafter, factor VIII is eluted from the chromatographic gel. Such methods are extremely complex, yet they give rise to highly pure factor VIII preparations. By the inventive use of an immobilized r-vWF having a high specific binding capacity and a high avidity for factor VIII, the complex of factor VIII and vWF occurring in plasma fractions can be dissociated, factor VIII binding to the immobilized vWF with higher avidity. In this way, highly pure factor VIII which is free from vWF can also be isolated from plasma fractions.

In addition to factor VIII, also other proteins having an affinity to vWF can be bound to immobilized r-VWF and isolated from complex mixtures, i.e., e.g., factor VIII hybride proteins, in particular factor VIII-heparin-cofactor II hybride proteins according to U.S. Ser. No. 08/558,107 or factor VIII-factor V-hybride protein according to WO 90/05530, or chimeric human/porcine factor VIII according to WO 94/11503, FVIII mutants, such as, e.g., factor VIII mutants having an Arg^{2 3 0 7}→Gln Substitution as described in AT 403 438 (factor VIII dB695-R2307Q), which, with a constant factor VIII:C-procoagulatory activity and vWF binding activity, exhibits a reduced inhibitor binding, von Willebrand factor-degrading enzymes, e.g. the vWF-specific depolymerase or vWF multimerase, respectively, or thrombocyte receptors, such as GPIIb/IIIa or GPIb/IX complex, whose pure preparation is of biochemical-analytical, diagnostic or therapeutic interest.

At present, the specific isolation of therapeutic proteins is carried out by means of the immunoaffinity chromatographic method. In doing so, the molecule to be isolated is bound to an immobilized monoclonal antibody (recovered from murine cells, as a rule), and washed free from impurities and subsequently is eluted in high purity. The drawback of using monoclonal antibodies consists in that the latter can deliver murine proteins into the preparation, which, in so far as the molecule to be bound is applied therapeutically, lead to side effects, such as, e.g., antibody formation to murine protein. By using an immobilized specific ligand of human origin or a molecule equivalent to the human protein, and by avoiding xenogenic antibodies, the immanent problem of a contamination by leakage of affinity columns is reduced in so far as the contamination possibly present in the preparation is also of human origin and thus the afore-said side effect, i.e. the formation of heterologous antibodies, is excluded. A further advantage consists in that by using a highly specific ligand which is not an antibody, non-specificities, as they may occur at times in antibodies, are excluded.

The recovery of the physiological binding proteins for vWF according to the method of the invention furthermore has the advantage that vWF exerts a stabilizer or carrier function for its binding partners. Even during their isolation and purification, the recovered proteins thus are protected from denaturing conditions which might occur during the elution. The products obtained thus are not only recovered in the afore-mentioned high yields as regards their antigenicity, but also as regards their activity or nativity, respectively.

A further, particularly preferred protein or protein complex, respectively, which can be purified with the vWF derivative of the invention, is the vWF multimerase which degrades the highly molecular forms of vWF to low-molecular variants (cf. AT 404 359 and AT 404 554). In this case, the multimerase can bind to the immobilized vWF, yet derivatisation prevents the degradation of the material of the invention.

In particular when isolating the vWF multimerase, elution can be particularly efficient with a buffer containing a chelating agent for metal ions, in particular EDTA.

The vWF derivative according to the invention is also suited for a possible extracorporeal immunoadsorption of antibodies directed against vWF. The formation of antibodies against vWF constitutes a pathologic condition which may occur as an autoimmune disease and leads to a blood coagulation defect with increased bleeding tendency or which may occur as a side effect of the treatment of patients with preparations containing vWF. The formation of a functionally inhibitory antibody renders a substitution therapy impossible or leads to drastic dosage increases to be able to maintain the hemostatic effect of the coagulation factor concentrate. In the past, in such instances, the circulating functional antibody against the coagulation factor had been removed by plasmapheresis or by extracorporeal immunoadsorption to proteins directed against IgG, e.g. protein A or protein G. A similar method is known from Nilsson et al. (Thromb. Haemostas. 70 (1993), 56-59) for removing the antibodies directed against factor VIII. In this instance, the plasma of the patient is pumped over a column comprising immobilized, recombinant FVIII so as to remove the FVIII-inhibitory antibodies from circulation. However, the ligand immobilized there was free from von Willebrand factor. Accordingly, no extracorporeal immunoadsorption of antibodies directed against vWF could be carried out.

A further application of the method of the invention for recovering antibodies resides in the preparative production of polyclonal or monoclonal anti-vWF antibodies for diagnostic purposes.

An elution buffer which preferably is used in the elution of antibodies from the inventive vWF derivative comprises an acidic pH, in particular a pH in the range of from 2 to 5.

One of the essential advantages of the present invention consists in that the purification of the proteins is possible from any starting material. Particularly preferred starting fractions are fractions which have been derivatized from a body liquid of a mammal or from a cell culture. Because of the inventive avidity of the chromatographic material, also fractions are preferred which comprise vWF and/or factor VIII/vWF-complex.

A preferred embodiment variant of the method according to the invention consists in that it is carried out by using the device of the invention with the vWF derivative contained therein.

The invention will now be explained in more detail by way of the following Examples and the drawing figures, to which, however, it is not to be restricted.

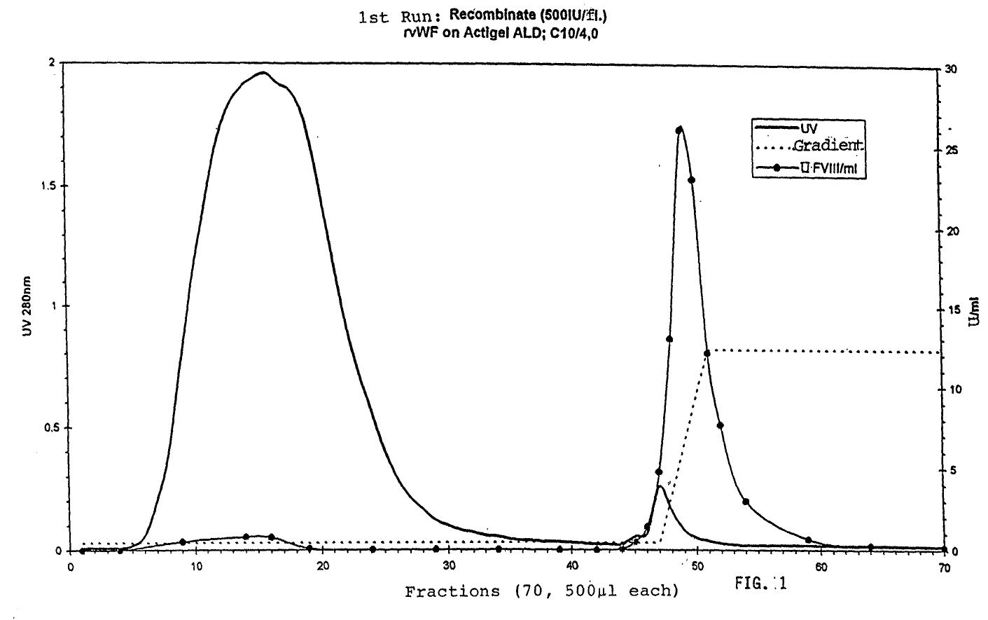

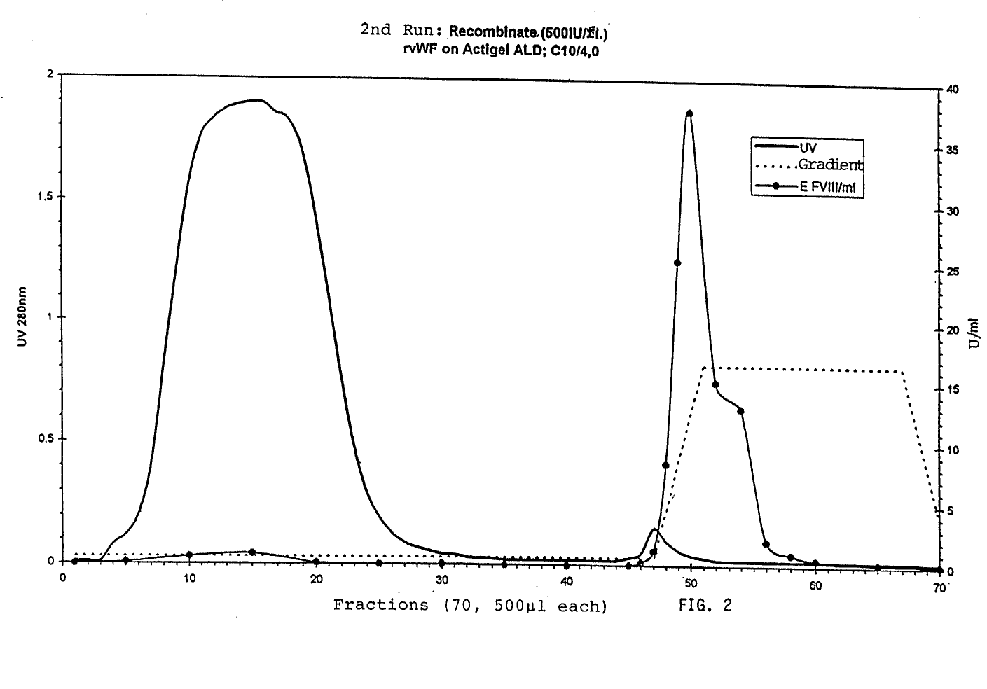

FIGS. 1 and 2 show the purification of recombinant factor VIII with the vWF derivative according to the invention.

### EXAMPLE 1

### Preparation of a Recombinant von Willebrand Factor from CHO Cells

A CHO cell clone producing recombinant von Willebrand factor is prepared as described in FEBS. Lett. 351 (1994), 345-348. By transfection with a vector encoding for the cDNA of human furin (van den Ouweland et al., Nucleic Acids Res. 18 (1990), 664), the cell line was made to co-express human furin. Such stable cell clones were fermented on a large scale on microcarriers in perfusion reactors (Blüml et al., in: Spier R E, Griffith J B, Berthold W, eds. Animal cell technology. Oxford, London: Butterworth-Heinemann, (1994) , 267-269).

Purification was carried out by a 2-stage, chromatographic method according to Thromb. Haemost. 73 (1995), 1160. The fraction desorbed by elution with saline was recovered and re-buffered by gel filtration over Sephadex G25 (Pharmacia) in a buffer containing 20 mM Tris-HCl, 150 mM NaCl, pH 7.5. Subsequently, the preparation was concentrated by ultra-concentration over an Amicon YM30 membrane (cut-off: 30,000 D) to a protein concentration of 3 mg/ml. The vWF concentration in this preparation amounted to 60 U of vWF antigen/mg protein. This preparation did not contain any factor VIII because serum or plasma components had been avoided at the preparation in cell culture and during purification.

### EXAMPLE 2

Immobilization of Recombinant von Willebrand Factor.

The preparation of recombinant vWF of Example 1 was diluted with a buffer containing 20 mM Tris-HCl, 150 mM NaCl, pH 7.5, to 1.5 mg/ml. A pre-activated gel suitable for affinity chromatography (Actigel, ALD-Superflow, Sterogene) was excessively pre-washed-with a buffer containing 20 mM Tris-HCl, 150 mM NaCl, pH 7.5. One volume portion of the pre-washed gel was admixed with 1.1 volume portions of the protein solution to be immobilized, and subsequently 0.15 volume portions of a solution of 0.1 M cyanoborohydride (NaCnBH₃) in 0.1 M phosphate buffer, pH 7.0, were admixed. The gel was suspended in this buffer by shaking, and under further shaking for 16 h incubated at room temperature. Subsequently, the gel was washed on a sinter suction filter with the 10-fold volume of a buffer containing 20 mM Tris-HCl, 150 mM NaCl, pH 7.5, and washed with the 5-fold volume of a buffer containing 20 mM Tris-HCl, 2M NaCl, pH 7.5. Then it was again equilibrated with 5 volume parts of the buffer, 20 mM Tris-HCl, 150 mM NaCl, pH 7.5, and the gel was transferred onto a chromatographic column having a dimension of diameter to gel bed height of 1:4. By determining the protein concentration in the solutions of the incubation supernatant of the vWF solution and of the affinity gel as well as the washing solutions separated on the sinter suction filter, a coupling rate of more than 90% of the protein used could be determined.

### EXAMPLE 3

Purification of Recombinant Factor VIII

A recombinant factor VIII preparation (Recombinante®, from Baxter) was reconstituted with 10 ml of Aqua. dest. This solution contained 50 IU FVIII/ml, 12 mg of human albumin/ml, 1.5 mg of polyethylene glycol 3350/ml, as well as traces of vWF in a histidine saline buffer at physiological pH. By gel filtration on Sephadex G25 (from Pharmacia) the low-molecular components were separated from this solution, and the factor VIII/vWF/albumin mixture was transferred into a buffer containing 20 mM Tris-HCl, pH 7.5. 7 ml of this solution were then applied to the column from Example 2 at a flow rate of 0.5 ml/min. Subsequently, at the same flow rate, it was washed with 10 ml of a buffer, 20 mM Tris-HCl, pH 7.5, and, furthermore, the vWF-bound FVIII fraction was eluted with 250 mM calcium chloride in the same buffer. During the entire chromatography which was carried out at room temperature, fractions of 500 μl were collected, and after passing through the column, the optical density was determined at 280 nm. In the fractions, subsequently the FVIII-content was determined by means of the chromogenic factor VIII test Immunochrom FVIII:C (from Immuno). The result can be taken from FIG. 1.

Analysis of the fractions shows that more than 90% of the protein were contained in the column effluent and in the wash solution, while only a small portion of the FVIII activity (less than 5%) was eluted with this protein fraction. The main amount of FVIII could be desorbed from the column in high purity by the elution with calcium chloride. Recovery of factor VIII was in a yield of more than 90%.

### EXAMPLE 4

### Reusability of the Affinity Gel

After completion of the first affinity chromatographic purification of recombinant factor VIII, the affinity gel in the column was washed excessively with a buffer, 20 mM Tris, pH 7.5. In the washing solution, vWF antigen was determined by means of ELISA (from Boehringer, Mannheim). No vWF:Ag could be measured, indicating a low leakage behavior of the affinity column. Affinity chromatographic purification of recombinant factor VIII on immobilized recombinant vWF was repeated as in Example 3 under identical conditions. The elution diagram can be taken from FIG. 2. Protein and activity elution profiles were comparable with the first affinity chromatographic run, within assay-related deviations. Thus, a good reusability of the affinity gel can be assumed.

### EXAMPLE 5

### Binding of anti-von Willebrand Factor Antibodies to Immobilized Recombinant von Willebrand Factor

A mouse-monoclonal antibody directed against vWF (MAb 03768/3, from Chemicon International, Inc.) having an IgG concentration of 7 mg/ml was rebuffered by gel filtration over Sephadex G25 (from Pharmacia) in a buffer, 20 mM Tris-HCl, 150 mM NaCl, pH 7.5. The protein concentration was adjusted by dilution with the same buffer to 0.5 mg/ml. 4 ml of this solution were pumped at a flow rate of 0.5 ml/min over the column containing immobilized recombinant vWF from Example 3, and the optical density in the effluent was determined at 280 mM. Subsequently, it was washed with 20 ml of the Tris-HCl buffer and, furthermore, with 10 ml of a Tris-HCl buffer containing 500 mM NaCl. In none of the washing steps indicated, any mentionable protein amounts could be eluted from the affinity column. Subsequently, it was eluted with a buffer, 100 mM Glycin-Hydrochloride, pH 2.2. By changing the pH, protein could be desorbed quantitatively from the column. Determination of the IgG content showed that from the 2 mg IgG used, 1.75 mg IgG were found again in the glycin eluate. This corresponds to a yield of 88%. After the elution step with the acidic buffer, the affinity column was flushed with 20 mM Tris-HCl buffer, pH 7.5, for further re-use.

### EXAMPLE 6

### Purification of a von Willebrand Factor Degrading Enzyme

The vWF-cleaving protease from human plasma described by Furlan et al. (Blood 87 (1996), 4223-4234), was pre-purified according to the method also described there via copper chelate affinity chromatography and subsequent hydrophobic interaction chromatography on butyl sepharose (from Pharmacia). The eluate of butyl sepharose was freeze-dried and taken up in distilled water after having been concentrated. Subsequently, it was re-buffered via Sephadex G25 (from Pharmacia) against a buffer, 20 mM Tris-HCl, 50 mM NaCl, 10 mM BaCl₂, 5 mM PEFA block (from Pentapharm, pH 7.5. 3 ml of this solution were applied over the column containing immobilized recombinant vWF from Example 2, at a flow rate of 0.5 ml/min. subsequently, it was washed with 10 ml of the same buffer, against which the protease fraction had been re-buffered, and the column was flushed with further 10 ml of a buffer containing 20 mM Tris-HCl, 50 mM NaCl, 20 mM EDTA, 5 mM PEFA block (from Pentapharm), pH 7.5. During the entire chromatography, fractions of 450 μl each were collected, 50 μl of a 1% aqueous solution of bovine serum albumin having been provided in the tubes of the fraction collector for each fraction so as to stabilize the vWF-degrading enzyme known to be labile. During chromatography, the UV absorption was measured at 280 nm, and the activity of the vWF-degrading enzyme was determined as follows. A preparation of the recombinant vWF from Example 1 was re-buffered in a buffer, 5 mM Tris-HCl, 1.5 M urea, containing 0.2% (W/V) bovine serum albumin, and brought to 0.4 vWF U/ml. Aliquots of the fractions to be examined of 100 μl were each admixed with 5 μl of a 50 mM aqueous PPACK solution and 12.5 μl of a 200 mM BaCl₂ solution and incubated at 37° C. for 5 min. Subsequently, the thus prepared sample was admixed 1+1 with the substrate (vWF) and dialysed for 15 h at 37° C. on a floating dialysis membrane (Millipore VSWP) according to the method of Marusky and Sergeant (Anal. Biochem. 105 (1980), 403) against a buffer containing 5 mM Tris-HCl, 1.5 M urea, pH 8.0. Subsequently, the dialysate was analysed for its vWF residual activity in an ELISA in which the collagen binding activity of vWF as well as its antigenicity are determined. To this end, 100 μl suspension of pepsin- digested type III collagen (from Southern Biotechnology) of human placenta, at a concentration of 2 μg/ml, were put into each well of a microtiter plate of polystyrene (96 well, Pierce Reactibind™/maleic acid anhydride-activated) and incubated at room temperature for 1 h. Subsequently, it was treated with 150 μl of block buffer each (SuperBlock™, from Pierce) for 30 minutes. Then, 100 μl each of various dilutions of the vWF-containing sample were applied (25-250 ng vWF/ml) and incubated with 100 μl of a solution of a peroxidase-conjugated anti-vWF antibody (Dakopatts P226, dilution 1:1000). Subsequently, 100 μl of substrate were added (Single Component TMB Peroxidase EIA Substrate, from Bio-Rad), and the color reaction was stopped after 1 min by 1+1 dilution with 0.18 M H₂ SO₄. Thereafter, the extinction at 450 nm was read by means of an ELISA reader, and the sample concentration determined relative to a dilution series of a standard. After each incubation step, it was washed 3 times each with 150 μl buffer (buffer corresponding to the respective next step).

### Buffer System Used

### Buffer 1 (for collagen coating and antibody dilution) 8 mM sodium phosphate, 135 mM NaCl, 2.6 mM KCl, pH 7.3

### Buffer 2 (for sample dilution): corresponds to buffer 1+0.05% Tween 20 and 1% bovine serum albumin, pH 7.3

The reciprocal value of the collagen binding activity is considered to be a direct measure for the enzyme activity of the vWF-degrading enzyme.

From the enzyme amount applied to the von Willebrand factor column, approximately 50% were found in the effluent and in the washing solutions, while approximately 50 of the activity could be desorbed from the column by elution with EDTA. However, since more than 95% of the protein were contained in the effluent and in the washing fractions of the chromatographic purification, the enzyme activity to be isolated could be enriched and purified by a factor 5-10 as compared to the starting material.

### EXAMPLE 7

### Purification of Plasmatic Factor VIII

A plasmatic factor VIII/von Willebrand factor concentrate (IMMUNATE; from Immuno) was reconstituted with 5 ml of distilled water. This solution contained 25 IU of factor VIII/ml and 10 U of vWF/ml (measured by means of the Ristocetin cofactor method) in a citrate-glycine-lysine buffer, pH 7.4. By gel filtration on Sephadex G25, the factor VIII/von Willebrand factor-complex was re-buffered against a buffer containing 20 mM Tris/HCl, pH 7.5. Subsequently, 3 ml of this solution were directly applied to the column of Example 2. The flow rate was 0.1 ml/min. Then it was washed with 30 ml of a 20 mM Tris/HCl buffer, pH 7.5, and thereafter it was eluted with a buffer containing 20 mM Tris/HCl and 250 mM CaCl₂. During the elution, fractions were collected, and the optical density was determined. In the fractions immediately after application of the elution buffer, an increase of the UV absorption of 280 nm by 10% could be measured, the desorbed protein being free from von Willebrand factor, measured in a von Willebrand factor ELISA (from Boehringer, Mannheim), while in the first ten eluate fractions a factor VIII content of approximately 0.2 U/ml could be found by determination with a chromogenic factor VIII assay, Immunochrom FVIII:C (from Immuno).

1. von Willebrand factor derivative (vWF derivative) comprised of recombinant von Willebrand factor (r-vWF), immobilized on a particulate carrier or a carrier gel.

2. vWF derivative according to claim 1, characterized in that the particulate carrier or the carrier gel is a chromatographic material.

3. vWF derivative according to claim 1 or 2, characterized in that the vWF derivative is free from blood coagulation factor VIII.

4. vWF derivative according to any one of claims 1 to 3, characterized in that the r-vWF is fixed to the particulate carrier or to the carrier gel by chemical binding.

5. vWF derivative according to any one of claims 1 to 4, characterized in that the r-vWF is derived from a rvWF fraction having a low primary hemostatic activity, in particular a low-molecular r-vWF fraction.

6. vWF derivative according to any one of claims 1 to 5, characterized in that the particulate carrier or the carrier gel is an organic polymer, in particular an organic polymer on carbohydrate basis.

7. vWF derivative according to any one of claims 1 to 6, characterized in that the r-vWF or the derivative, respectively, is virus-inactivated.

8. vWF derivative according to any one of claims 1 to 7, characterized in that it is present in storage-stable form, in particular as a lyophilisate.

9. Device comprising a container and a vWF derivative according to any one of claims 1 to 8 contained therein, the container having an inlet aperture and an outlet aperture which are designed to be suitable for passing liquids therethrough.

10. A device according to claim 9, characterized in that the container is designed as an affinity column.

11. A method of isolating proteins which bind to vWF, characterized by the following steps:
providing a fraction containing proteins which bind to vWF,
contacting the fraction with a vWF derivative according to any one of claims 1 to 8, wherein the proteins become bound to the vWF derivative,
separating non-bound components, and
eluting the proteins from the vWF derivative.

12. A method according to claim 11, characterized in that the proteins are eluted in concentrated form from the vWF derivative.

13. A method according to claim 11 or 12, characterized in that a fraction comprising a biologically active protein with factor VIII activity is provided and this protein is isolated.

14. A method according to any one of claims 11 to 13, characterized in that it is eluted with a calcium ion-containing buffer.

15. A method according to claim 11 or 12, characterized in that a fraction comprising a vWF multimerase is provided and this protein is isolated.

16. A method according to any one of claims 11, 12 or 15, characterized in that it is eluted with a buffer comprising a chelating agent for metal ions, in particular EDTA.

17. A method according to claim 11 or 12, characterized in that a fraction comprising antibodies to vWF is provided and these proteins are isolated.

18. A method according to any one of claims 11, 12 or 17, characterized in that it is eluted with a buffer having an acidic pH, in particular in the range of from pH 2 to 5.

19. A method according to any one of claims 11 to 18, characterized in that a fraction is provided which is derived from a body liquid of a mammal or from a cell culture.

20. A method according to any one of claims 11 to 19, characterized in that a fraction comprising vWF and/or the factor VIII/vWF-complex is provided.

21. A method according to any one of claims 11 to 20, characterized in that the proteins are recovered in a yield of at least 80%.

22. A method according to any one of claims 11 to 21, characterized in that it is carried out by using a device according to claim 9 or 10.
